(19) United States
(12) Patent Application Publication
(10) Pub. No.: US 2024/0415799 A1
(43) Pub. Date: Dec. 19, 2024

(54) COMPOSITION FOR LIVING BODY

(71) Applicant: FUJIFILM Corporation, Tokyo (JP)

(72) Inventors: Haruki TOMIKAWA, Kanagawa (JP); Toshihide YOSHITANI, Kanagawa (JP)

(73) Assignee: FUJIFILM Corporation, Tokyo (JP)

(21) Appl. No.: 18/815,847

(22) Filed: Aug. 27, 2024

Related U.S. Application Data
(63) Continuation of application No. PCT/JP2023/008501, filed on Mar. 7, 2023.

(30) Foreign Application Priority Data
Mar. 28, 2022 (JP) 2022-051370

(51) Int. Cl. A61K 31/231; A61K 31/045; A61K 31/685; A61K 45/06

(52) U.S. Cl. CPC A61K 31/231; A61K 31/045; A61K 31/685; A61K 45/06

(57) ABSTRACT

In the present invention, there is provided a composition for a living body, which has excellent spreadability on an object, has a high film formation rate in a case of being brought into contact with water to form a film, and has excellent strength and excellent controlled release characteristics of the film. The present invention relates to a composition for a living body, containing a neutral acyl lipid, a phospholipid, an alcohol having 4 or less carbon atoms or a polyalkylene oxide, and a physiologically active substance, in which a content of a neutral diacyl lipid in the neutral acyl lipid is more than 0% by mass and 50% by mass or less, and a mass ratio of a content of the neutral acyl lipid to a content of the phospholipid is 54/46 to 1/99.

### CROSS-REFERENCE TO RELATED APPLICATIONS

This application is a Continuation of PCT International Application No. PCT/JP2023/008501 filed on Mar. 7, 2023, which claims priority under 35 U.S.C. § 119(a) to Japanese Patent Application No. 2022-051370 filed on Mar. 28, 2022. The above applications are hereby expressly incorporated by reference, in their entirety, into the present application.

### BACKGROUND OF THE INVENTION

### 1. Field of the Invention

The present invention relates to a composition for a living body.

### 2. Description of the Related Art

In the related art, there is a demand for suppressing pain caused by inflammation or the like in a mucous membrane or a skin.

For example, in a cancer patient, cancer treatment affects the oral mucosa, which easily causes stomatitis. In anti-cancer drug treatment, in radiation therapy for head and neck cancer (cancer in the range from the head to the neck) in a case where a drug that easily causes stomatitis is administered, when radiation is directly applied to the mucous membrane of the mouth, stomatitis is inevitable. The pain of stomatitis is so strong that it is difficult to eat a meal by mouth.

As a symptomatic treatment for stomatitis, a patch (for example, Aphthaseal® 25 g, manufactured by Taisho Pharmaceutical Co., Ltd., active ingredient: triamcinolone acetonide) that is directly attached to the affected part, an ointment (for example, Dexaltin oral ointment for oral cavity, manufactured by Nippon Kayaku Co., Ltd., active ingredient: dexamethasone) that is applied to the affected part, and a spray agent (for example, Salcoat® capsule for oral spray 50 g, manufactured by TEIJIN PHARMA LIMITED, active ingredient: beclomethasone propionate ester) that is sprayed on the affected part are mentioned.

However, when eating a meal by mouth, the patch attached to the affected part may be peeled off, or the ointment or the spray agent applied to the affected part may be lost, and thus the pain of stomatitis cannot be suppressed.

On the other hand, as for the sustainability of the action of the composition to be locally applied, a preformulation in the form of a low viscosity mixture (molecular solution or the like) consisting of an amphiphilic component and at least one physiologically active substance optionally contained, in which in a case where the preformulation is exposed to an aqueous fluid such as body fluid, at least one phase transition is caused to form a bioadhesive matrix, is known.

For example, JP5144277B describes a formulation containing a low viscosity mixture of a) at least one neutral diacyl lipid containing at least 50% glyceryl dioleate, b) at least one phospholipid consisting of at least 50% phosphatidylcholine, and c) 2% to 30% by weight at least one biocompatible organic solvent containing ethanol, in which a weight ratio of the component a) to the component b) is 85:15 to 30:70, at least one bioadhesive liquid crystal phase structure can be formed by contact with an aqueous fluid and/or a body surface, and a viscosity is 0.1 to 5,000 mPas at 20° C.

### SUMMARY OF THE INVENTION

On the other hand, in a case where the composition for a living body to be applied to a living body contains a physiologically active substance (formulation), various characteristics are required.

Specifically, in a case where the composition for a living body is applied to an object and is brought into contact with water in the atmosphere or the like to form a film, it is required that a formation rate of the film is high. In a case where the liquid crystal film (film exhibiting a liquid crystal phase) is formed by contact of the composition for a living body with water, a formation rate of the film corresponds to a rate at which the liquid crystal phase is formed.

In addition, it is also required that the composition for a living body has excellent spreadability in a case of applying the composition for a living body to an object. Furthermore, it is also required that the formed film itself has excellent strength and that the controlled release characteristics of the physiologically active substance from the formed film (controlled release characteristics of the film) is excellent.

As a result of studying the characteristics of the preformulation of the composition described in JP5144277B, the present inventors have found that all of the above-described specific characteristics cannot be simultaneously satisfied, and in particular, the controlled release characteristics of the formed film is deteriorated, and further improvement is required.

An object of the present invention is to provide a composition for a living body, which has excellent spreadability on an object, has a high film formation rate in a case of being brought into contact with water to form a film, and has excellent strength and excellent controlled release characteristics of the formed film.

As a result of intensive examination on the above-described problem, the present inventors found that the above-described problem can be solved by the following configurations.

[1] A composition for a living body, comprising a neutral acyl lipid, a phospholipid, an alcohol having 4 or less carbon atoms or a polyalkylene oxide, and a physiologically active substance, in which a content of a neutral diacyl lipid in the neutral acyl lipid is more than 0% by mass and 50% by mass or less, and a mass ratio of a content of the neutral acyl lipid to a content of the phospholipid is 54/46 to 1/99.

[2] The composition for a living body according to [1], in which the mass ratio is 54/46 to 30/70.

[3] The composition for a living body according to [1] or [2], in which the mass ratio is 49/51 to 36/64.

[4] The composition for a living body according to any one of [1] to [3], in which the mass ratio is 49/51 to 41/59.

[5] The composition for a living body according to any one of [1] to [4], in which the neutral acyl lipid further contains a neutral monoacyl lipid, and a content of the neutral monoacyl lipid in the neutral acyl lipid is 40% to 90% by mass.

[6] The composition for a living body according to [5], in which the neutral monoacyl lipid contains glyceryl monooleate.

[7] The composition for a living body according to any one of [1] to [6], in which the phospholipid contains an ionic phospholipid.

[8] The composition for a living body according to any one of [1] to [7], in which the phospholipid contains phosphatidylcholine and a content of the phosphatidylcholine in the phospholipid is at least 50% by mass.

[9] The composition for a living body according to any one of [1] to [8], in which a content of the alcohol having 4 or less carbon atoms or the polyalkylene oxide is 0.5 to 10 parts by mass with respect to 100 parts by mass of a total content of the neutral acyl lipid and the phospholipid.

[10] The composition for a living body according to any one of [1] to [9], in which a content of water is 0% by mass or more and 10% by mass or less with respect to a total amount of a remainder obtained by removing the physiological active substance from the composition for a living body.

[11] The composition for a living body according to any one of [1] to [10], in which a reversed hexagonal columnar phase is formed by water absorption or moisture absorption.

[12] The composition for a living body according to any one of [1] to [11], in which the composition for a living body is for percutaneous absorption or mucous membrane absorption.

According to the present invention, it is possible to provide a composition for a living body, which has excellent spreadability on an object, has a high film formation rate in a case of being brought into contact with water to form a film, and has excellent strength and excellent controlled release characteristics of the formed film.

### BRIEF DESCRIPTION OF THE DRAWINGS

FIG. 1 is photographs showing gelatin gels after controlled release of cyanocobalamin from the composition for a living body of Examples 1 to 4 and Comparative Examples 1 to 3 in the evaluation of the controlled release characteristics of the present invention.

### DESCRIPTION OF THE PREFERRED EMBODIMENTS

Hereinafter, the present invention will be described in detail.

The description of the configuration requirements described below is made on the basis of representative embodiments of the present invention, but it should not be construed that the present invention is limited to those embodiments.

Hereinafter, meaning of each description in the present specification will be explained.

In the present specification, the range indicated by using “to” means a range including both ends before and after “to”. For example, a range indicated by “A to B” includes A and B.

In the present specification, the substrate refers to a remainder obtained by removing a physiologically active substance from the composition for a living body according to the embodiment of the present invention.

### [Composition for Living Body]

The composition for a living body according to the embodiment of the present invention is a composition for a living body, containing a neutral acyl lipid containing a neutral diacyl lipid, a phospholipid, an alcohol having 4 or less carbon atoms or a polyalkylene oxide, and a physiologically active substance.

Each component contained in the composition for a living body according to the embodiment of the present invention will be described below.

### [Neutral Acyl Lipid]

The neutral acyl lipid means an electrically neutral acyl lipid. That is, the neutral acyl lipid does not include a cation moiety and an anion moiety. The acyl lipid means a lipid containing an acyl group.

### [Neutral Diacyl Lipid]

In the present invention, the neutral acyl lipid contains a neutral diacyl lipid. The neutral acyl lipid can further contain at least one or both of a neutral monoacyl lipid and a neutral triacyl lipid, in addition to the neutral diacyl lipid.

### [Content of Neutral Diacyl Lipid]

In the present invention, the content of the neutral diacyl lipid in the neutral acyl lipid is, with respect to the total amount of the neutral acyl lipid, more than 0% by mass (exceeding 0% by mass) and 50% by mass or less, and from the viewpoint that the effects of the present invention (particularly, the spreadability, the strength of the film, and the controlled release characteristics) are more excellent, is preferably 10% to 50% by mass and more preferably 20% to 50% by mass.

### (Neutral Monoacyl Lipid)

In a case where the neutral acyl lipid further contains a neutral monoacyl lipid, from the viewpoint that the effects of the present invention (particularly, the spreadability, the strength of the film, and the controlled release characteristics) are more excellent, the content of the neutral monoacyl lipid in the neutral acyl lipid is, with respect to the total amount of the neutral acyl lipid, preferably 40% to 90% by mass and more preferably 40% to 70% by mass.

The neutral monoacyl lipid preferably contains glyceryl monooleate.

### (Neutral Triacyl Lipid)

In the present invention, from the viewpoint that the effects of the present invention (particularly, the spreadability, the strength of the film, and the controlled release characteristics) are more excellent, the content of the neutral triacyl lipid in the neutral acyl lipid is, with respect to the total amount of the neutral acyl lipid, preferably 0% to 10% by mass and more preferably 1% to 8% by mass.

The number of carbon atoms of the acyl group of the neutral acyl lipid is not particularly limited; however, the acyl group preferably has 6 to 32 carbon atoms and more preferably 16 to 22 carbon atoms.

The hydrocarbon group as the acyl group, excluding a carbonyl group, is preferably a saturated or unsaturated chain-type hydrocarbon group having 5 to 31 carbon atoms, and more preferably a saturated or unsaturated chain-type hydrocarbon group having 15 to 21 carbon atoms. Specific examples thereof include CH₃(CH₂)₁₄—, CH₃(CH₂)₇CH═CH(CH₂)₇—, and CH₃(CH₂)₄(CH═CHCH₂)₂(CH₂)₆—; however, the examples are not limited thereto.

In a case where a neutral acyl lipid has two or more acyl groups in one molecule, the kinds of acyl groups may be the same or different from each other.

Examples of the neutral acyl lipid include glycerol, diglycerol, sugar (for example, inositol), and a lipid obtained by forming an ester bond between a polyol such as succinic acid and a fatty acid. Among the above, an acyl glycerol is preferable and a glyceryl oleate is more preferable.

Examples of the acyl glycerol include monoacyl glycerol, diacyl glycerol, and triacyl glycerol.

In addition, examples of the glyceryl oleate include glyceryl monooleate, glyceryl dioleate, and glyceryl trioleate.

Here, the monoacyl glycerol and the glyceryl monooleate correspond to one aspect of the neutral monoacyl lipid described above, and the diacyl glycerol and the glyceryl dioleate correspond to one aspect of the neutral diacyl lipid described above, and the triacyl glycerol and the glyceryl trioleate correspond to one aspect of the neutral triacyl lipid.

In the present specification, the content proportions of the neutral monoacyl lipid, the neutral diacyl lipid, and the neutral triacyl lipid in the neutral acyl lipid are obtained from measurement with a high performance liquid chromatography (HPLC) method.

### HPLC Measurement Conditions

Column: Cadenza CD-C18 (4.6 mm×300 mm) (manufactured by Imtakt Corporation)
Eluent: water and tetrahydrofuran
Flow rate: 1.0 mL/min
Detection: Corona Charged Aerosol Detector (Corona CAD)
Column temperature: 50° C.

### [Phospholipid]

The composition for a living body according to the embodiment of the present invention contains a phospholipid.

The phospholipid is not particularly limited as long as it has a phosphate ester structure in the molecular structure thereof, however, a glycerophospholipid having glycerin as a skeleton and a sphingophospholipid having sphingosine as a skeleton are typical.

In both a case of the phospholipid being a glycerophosphoric acid and a case of the phospholipid being a sphingophospholipid, an acyl group derived from a fatty acid is contained in the molecule.

The number of carbon atoms of the acyl group of the phospholipid is not particularly limited; however, the acyl group preferably has 12 to 22 carbon atoms and more preferably 16 to 18 carbon atoms.

The hydrocarbon group as the acyl group, excluding a carbonyl group, is preferably a saturated or unsaturated chain-type hydrocarbon group having 11 to 21 carbon atoms, and more preferably a saturated or unsaturated chain-type hydrocarbon group having 15 to 17 carbon atoms. Specific examples of the hydrocarbon group include CH₃(CH₂)₁₄—, CH₃(CH₂)₇CH═CH(CH₂)₇—, and CH₃(CH₂)₄(CH═CHCH₂)₂(CH₂)₆—; however, the examples are not limited thereto.

In a case where a phospholipid has two or more acyl groups in one molecule, the kinds of acyl groups may be the same or different from each other.

Specific examples of the phospholipid include phosphatidylcholine. The acyl group of the phosphatidylcholine is preferably an acyl group derived from palmitic acid (CH₃(CH₂)₁₄COOH), oleic acid (CH₃(CH₂)₇CH═CH(CH₂)₇COOH), or linoleic acid (CH₃(CH₂)₄(CH═CHCH₂)₂(CH₂)₆COOH). Specific examples of the phosphatidylcholine include a PO phosphatidylcholine (a phosphatidylcholine having palmitic acid at the first position (the α position), oleic acid at the second position (the R position), and choline at the third position (the γ position)), a DL phosphatidylcholine (a phosphatidylcholine having linoleic acid at the first position (the α position), linoleic acid at the second position (the R position), and choline at the third position (the γ position)), and a dipalmitoyl phosphatidylcholine.

From the viewpoint that the water absorption rate of the composition for a living body according to the embodiment of the present invention can be improved or a columnar phase having a large domain size can be formed, the phospholipid preferably contains an ionic phospholipid. It is considered that the improvement of the water absorption rate of the composition for a living body and the like contributes to the further improvement of at least one of the film formation rate or the strength of the film. The above-described water absorption rate refers to a rate at which the composition for a living body according to the embodiment of the present invention absorbs water or moisture.

Examples of the ionic phospholipid include a phospholipid having a cationic moiety and an anionic moiety in one molecule, and specific examples thereof include phosphatidylcholine. Phosphatidylcholine has, as a cationic moiety, N⁺ derived from choline and P—O⁻ derived from phosphoric acid.

In a case where the phospholipid contains phosphatidylcholine, the content of the phosphatidylcholine in the phospholipid is preferably at least 50% by mass with respect to the total amount of the phospholipid. The upper limit of the content of phosphatidylcholine with respect to the total amount of phospholipids is not particularly limited, and examples thereof is 99% by mass.

### [Mass Ratio of Content of Neutral Acyl Lipid to Content of Phospholipid]

In the present invention, the mass ratio (neutral acyl lipid/phospholipid) of the content of the neutral acyl lipid to the content of the phospholipid is 54/46 to 1/99.

From the viewpoint that the effect of the present invention is more excellent, the above-described mass ratio is preferably 54/46 to 30/70, more preferably 49/51 to 36/64, and still more preferably 49/51 to 41/59.

### (Total Content of Neutral Acyl Lipid and Phospholipid)

The total content of the neutral acyl lipid and the phospholipid is preferably 77% to 99.5% by mass and more preferably 93% to 98% by mass with respect to the total amount of the substrate (remainder obtained by removing the physiologically active substance from the composition for a living body according to the embodiment of the present invention).

### [Alcohol Having 4 or Less Carbon Atoms or Polyalkylene Oxide]

The composition for a living body according to the embodiment of the present invention contains an alcohol having 4 or less carbon atoms or a polyalkylene oxide.

The composition for a living body according to the embodiment of the present invention has excellent spreadability by containing an alcohol having 4 or less carbon atoms or a polyalkylene oxide.

The alcohol having 4 or less carbon atoms is not particularly limited as long as it is a compound having biocompatibility. The same applies to the polyalkylene oxide.

An alcohol having 4 or less carbon atoms can function as a solvent. The same applies to the polyalkylene oxide.

### [Alcohol Having 4 or Less Carbon Atoms]

The alcohol having 4 or less carbon atoms is a compound in which a hydroxy group is bonded to an aliphatic hydrocarbon group having 3 or less carbon atoms. The number of hydroxy groups included in one molecule of the alcohol having 4 or less carbon atoms is preferably 1 or 2.

Examples of the alcohol having 4 or less carbon atoms include monohydric alcohols such as ethanol; and dihydric alcohols such as propylene glycol and 1,3-butylene glycol.

### [Polyalkylene Oxide]

The polyalkylene oxide is a polymer having an oxyalkylene group as a repeating unit.

Terminals of the polyalkylene oxide may form a hydroxy group.

The polyalkylene oxide may have one or a plurality of hydroxy groups per molecule.

Examples of the polyalkylene oxide include polyoxyethylene polyol, polyoxypropylene polyol, and polyoxyethyleneoxypropylene polyol.

### (Content of Alcohol Having 4 or Less Carbon Atoms and the Like)

A content of the alcohol having 4 or less carbon atoms or the polyalkylene oxide is preferably 0.5 to 10 parts by mass and more preferably 2.0 to 8.0 parts by mass with respect to 100 parts by mass of the total content of the neutral acyl lipid and the phospholipid.

In a case where the composition for a living body according to the embodiment of the present invention contains the alcohol having 4 or less carbon atoms and the polyalkylene oxide, the total content of the alcohol having 4 or less carbon atoms and the polyalkylene oxide can be 1.0 to 20 parts by mass with respect to 100 parts by mass of the total content of the neutral acyl lipid and the phospholipid.

### [Physiologically Active Substance]

The composition for a living body according to the embodiment of the present invention contains a physiologically active substance.

The physiological active substance is released on the surface of a living body by applying the composition for a living body according to the embodiment of the present invention to the surface of a living body, and is absorbed into the body through the skin, the mucous membrane, or the like. The physiologically active substance may be any substance (active ingredient) to be administered to a living body. The physiologically active substance is not a lipid. The physiologically active substance may be an organic compound or an inorganic compound. The physiologically active substance may be water-soluble or fat-soluble (lipophilic, water-insoluble, or low water-soluble). The physiologically active substance may be a protein, a peptide, an amino acid, a nucleic acid, a vitamin, a hormone, an enzyme, a mineral, or the like, but is not limited thereto. The physiologically active substance may be, for example, an anti-cancer drug, an immunosuppressive agent, an analgesic (for example, a non-opioid analgesic or an opioid analgesic such as morphine), an anti-inflammatory agent, an antiallergic agent (such as tranilast), a steroid drug (such as triamcinolone acetonide), an anti-obesity drug, an antidiabetic drug, an antibiotic, an antifungal agent, an antiviral agent, a vasodilator, an anesthetic, a smoking-cessation aid (nicotine or the like), an antipsychotic drug, an antihypertensive agent, a cardiotonic agent, a β-blocking agent, an antianemic agent, an antihyperlipidemic agent, a bronchodilator, a dementia therapeutic agent, a brain and central nervous system disease therapeutic agent for Alzheimer's disease, Parkinson's disease, cerebrovascular disease, or brain tumor, a chronic obstructive pulmonary disease (COPD) therapeutic agent, a glaucoma therapeutic agent, a cataract therapeutic agent, an age-related macular degeneration therapeutic agent, an overactive bladder therapeutic agent, an attention deficit/hyperactivity disorder therapeutic agent, a hormone agent, a vaccine or the like, but is not limited thereto.

### (Content of Physiologically Active Substance)

A content of the physiologically active substance is not limited, but with respect to the total amount of the composition for a living body according to the embodiment of the present invention, may be typically 0.0001% by mass or more, for example, 0.0001% to 10% by mass, 0.0005% to 5% by mass, 0.0005% to 1% by mass, 0.001% to 5% by mass, 0.001% to 1% by mass, 0.001% to 0.1% by mass, 0.001% to 0.05% by mass, 0.001% to 0.01% by mass, 0.01% to 5% by mass, 0.01% to 1% by mass, 0.01% to 0.1% by mass, 0.05% to 1% by mass, or 0.1% to 0.5% by mass.

### (Content of Substrate)

The content of the substrate is not limited, but the content of the base material can be an amount obtained by subtracting the content of the physiologically active substance from the total amount of the composition for a living body according to the embodiment of the present invention.

### (Other Components)

The composition for a living body according to the embodiment of the present invention can further contain a quaternary ammonium salt (excluding phosphatidylcholine) and water.

### (Quaternary Ammonium Salt)

The quaternary ammonium salt is an ionic compound consisting of a polyatomic ion having a positive charge, which is represented by a molecular formula NR₄⁺ (quaternary ammonium cation), and an anion. In NR₄⁺, R's each independently represent an alkyl group or an aryl group, and a plurality of R's may be the same as or different from each other. The anion is not particularly limited.

Examples of the quaternary ammonium salt include dioleoyloxytrimethylammonium propane chloride (DOTAP, N-[1-(2,3-dioleoyloxy)propyl]-N,N,N-trimethylammonium chloride), dioctadecenyltrimethylammonium propane chloride (DOTMA, N-[1-(2,3-dioleoyloxy)propyl)]-N,N,N-trimethylammonium chloride), didecyldimethylammonium chloride, didecyldimethylammonium bromide, dilauryldimethylammonium chloride, dicetyldimethylammonium chloride, dicetyldimethylammonium bromide, distearyldimethylammonium chloride, distearyldimethylammonium bromide, dioleyldimethylammonium chloride, dibehenyldimethylammonium chloride, dibehenyldimethylammonium bromide, dipalmitoylethyl hydroxyethylmonium metosulfate, and distearoylethyl hydroxyethylmonium metosulfate.

The composition for a living body according to the embodiment of the present invention may or may not contain water.

The content of the water is preferably 0% by mass or more and 10% by mass or less, and more preferably 0% by mass with respect to the total amount of the remainder (substrate) obtained by removing the physiologically active substance from the composition for a living body according to the embodiment of the present invention. In a case where the content of the water is 0% by mass with respect to the total amount of the above-described remainder (substrate), the composition for a living body according to the embodiment of the present invention can be a composition not containing the water.

From the viewpoint of ensuring the transparency of the obtained film, it is preferable to exclude the aspect containing the carboxyvinyl polymer from the composition for a living body according to the embodiment of the present invention. That is, it is preferable that the composition for a living body according to the embodiment of the present invention does not contain the carboxyvinyl polymer. The carboxyvinyl polymer is a vinyl polymer having a carboxy group or a salt thereof. Examples of the carboxyvinyl polymer include a polymer having a repeating unit derived from acrylic acid and/or methacrylic acid as a main chain. The carboxyvinyl polymer may be crosslinked. For details of the carboxyvinyl polymer, WO2020/059543A can be referred to. The aspect containing the carboxyvinyl polymer may further include or may not include a amphiphilic block polymer, which will be described later.

In addition, it is preferable to exclude, from the composition for a living body of the present invention, an aspect containing an amphiphilic block polymer which consists of a hydrophilic segment and a hydrophobic segment and has a difference in C log P value between the hydrophilic segment and the hydrophobic segment of more than 1.00. That is, it is preferable that the composition for a living body according to the embodiment of the present invention does not contain the above-described amphiphilic block polymer. The amphiphilic block polymer is the same as “the amphiphilic block polymer which consists of a hydrophilic segment and a hydrophobic segment and has a difference in C log P value between the hydrophilic segment and the hydrophobic segment of more than 1.00” described in WO2020/202926A. The aspect including the above-described amphiphilic block polymer may further include or may not include the above-described carboxyvinyl polymer.

### (Method for Producing Composition for Living Body)

Examples of a manufacturing method of the composition for a living body according to the embodiment of the present invention include a method of mixing the neutral acyl lipid, the phospholipid, the alcohol having 4 or less carbon atoms or the polyalkylene oxide, the physiologically active substance, and the other components which can be used as necessary. In the above-described mixing, the content of the neutral diacyl lipid may be more than 0% by mass and 50% by mass or less with respect to the total amount of the neutral acyl lipids, and a mass ratio of the content of the neutral acyl lipid to the content of the phospholipid may be 54/46 to 1/99.

The method of mixing is not particularly limited, and a conventionally known method can be used.

### (Liquid Crystal Phase)

The composition for a living body according to the embodiment of the present invention can form a liquid crystal phase by being brought into contact with water. In the present invention, as described above, the composition for a living body according to the embodiment of the present invention forms a liquid crystal phase, thereby the composition for a living body according to the embodiment of the present invention forms a film.

The composition for a living body according to the embodiment of the present invention can form quickly a liquid crystal phase in a short time even in a case of being brought in contact with a small amount of water, for example, water (moisture) in the atmosphere.

A thickness of the liquid crystal phase (film) formed of the composition for a living body according to the embodiment of the present invention is not particularly limited, but can be, for example, 0.1 m to 1 mm.

In addition, the composition for a living body according to the embodiment of the present invention can be more strongly adhered to an object by being brought into contact with water.

### (Water)

Examples of the water to be brought into contact with the composition for a living body according to the embodiment of the present invention include water (moisture) in the atmosphere, water (moisture) in exhalation, pure water, and water contained in an aqueous fluid other than the water. Examples of the aqueous fluid other than water include saliva, tissue fluid, blood, and lymph.

In a case of using the composition for a living body according to the embodiment of the present invention, the amount of water to be brought into contact with the composition for a living body according to the embodiment of the present invention is not particularly limited, but is preferably 1,000% by mass or less and more preferably 500% by mass or less with respect to the total mass of the composition for a living body according to the embodiment of the present invention. The lower limit of the amount of the water with respect to the total mass of the composition for a living body according to the embodiment of the present invention, which is used in a case of being brought into contact with the composition for a living body according to the embodiment of the present invention, is not particularly limited, and examples thereof include more than 1% by mass.

A temperature at which the composition for a living body according to the embodiment of the present invention is brought into contact with water is not particularly limited, but is preferably 20° C. to 40° C. and more preferably 35° C. to 40° C.

The liquid crystal phase which can be formed by bringing the composition for a living body according to the embodiment of the present invention into the contact with water is not particularly limited, and is any one selected from the group consisting of a reversed hexagonal columnar (H2) phase, a hexagonal columnar (H1) phase, a lamellar (La) phase, a sponge (V2) phase, a bicontinuous cubic (L3) phase, and a mixed state of two or more thereof in many cases. The above-described liquid crystal phase preferably has a reversed hexagonal columnar phase (W/O hexagonal columnar phase) or a hexagonal columnar (H1) phase (O/W hexagonal columnar phase).

The composition for a living body according to the embodiment of the present invention preferably forms a reversed hexagonal columnar (H2) phase by water absorption or moisture absorption, and more preferably forms a reversed hexagonal columnar (H2) phase by moisture absorption.

The moisture absorption refers to the absorption of moisture. Examples of the moisture include water in the atmosphere and water in the exhalation.

In the present specification, the water absorption refers to the absorption of water (excluding moisture). Examples of the water (excluding moisture) include pure water and the aqueous fluid other than the water described above.

### (Application of Composition for Living Body)

The composition for a living body according to the embodiment of the present invention means a material which can be used for a living body. In a living body, the composition for a living body according to the embodiment of the present invention can be used, for example, for the purpose of assisting or repairing a part that does not exhibit an original function due to an injury, a disease, or the like (for example, an object such as a skin or a mucous membrane, the same applies to “portion” hereinafter), and a part where the function is decreased.

Since the composition for a living body according to the embodiment of the present invention contains a physiologically active substance, it can function as a preparation. By applying the composition for a living body according to the embodiment of the present invention to a living body surface, a physiologically active substance is released from the composition for a living body according to the embodiment of the present invention to the surface of the living body and can be absorbed into the body through the skin, mucous membrane, or the like.

The composition for a living body according to the embodiment of the present invention can be preferably used for a percutaneous absorption or a mucous membrane absorption (particularly, for an oral mucous membrane absorption).

### (Method for Using Composition for Living Body)

Examples of the method for using the composition for a living body according to the embodiment of the present invention include a method in which the composition for a living body according to the embodiment of the present invention is disposed on a portion having the symptoms described above, and then the composition for a living body according to the embodiment of the present invention is brought into contact with water.

In a case where the composition for a living body according to the embodiment of the present invention is used on the skin, for example, the composition for a living body according to the embodiment of the present invention may be applied onto the skin in the atmosphere, and water or an aqueous solution containing water may be added to the composition for a living body according to the embodiment of the present invention as necessary.

The composition for a living body according to the embodiment of the present invention on the skin can form a liquid crystal phase by being brought into contact with, for example, any of water in the atmosphere, water sprayed by spraying, the above-described aqueous fluid, and exudate from the skin.

In a case where the composition for a living body according to the embodiment of the present invention is used for a mucous membrane, the composition for a living body according to the embodiment of the present invention may be disposed on the mucous membrane, and water or an aqueous solution containing water may be added to the composition for a living body according to the embodiment of the present invention as necessary.

The composition for a living body according to the embodiment of the present invention on the mucous membrane can form a liquid crystal phase by being brought into contact with, for example, any of water in the atmosphere, water in the exhalation, water sprayed by spraying, the above-described aqueous fluid, or water ingested.

In particular, in a case where the composition for a living body according to the embodiment of the present invention is applied to an oral mucous membrane, since the composition for a living body according to the embodiment of the present invention is adhered (applied) to the oral mucous membrane and thus brought into contact with any of water in the atmosphere, water in the exhalation, or water in the saliva to form the liquid crystal phase, the handling is simple. In addition, in a case where the amount of saliva is small, it is sufficient to supply the moisture by spraying water or artificial saliva after attaching the composition for a living body according to the embodiment of the present invention to the oral mucous membrane.

### (Phase Transition)

In the composition for a living body according to the embodiment of the present invention, the liquid crystal phase may undergo a phase transition by further absorbing water after absorbing moisture. It is preferable that the liquid crystal phase of the composition for a living body according to the embodiment of the present invention undergoes a phase transition, thereby the effect of the present invention (particularly the controlled release characteristics) is more excellent. Examples of the above-described phase transition include a phase transition from a reversed hexagonal columnar (H2) phase to a hexagonal columnar (H1) phase. Specifically, as described above, examples thereof include an aspect in which the moisture in a small amount of water (moisture) such as water in the atmosphere and water in exhalation is absorbed to form a liquid crystal phase (for example, a reversed hexagonal columnar (H2) phase), and then a larger amount of water than water (moisture) such as in the atmosphere from the air (moisture), such as water sprayed by spraying, artificial saliva, an aqueous fluid, exudate from a skin, and water ingested is absorbed to cause the above-described liquid crystal phase to undergo a phase transition to another liquid crystal phase (for example, a hexagonal columnar (H1) phase).

From the viewpoint of the effect of the present invention (particularly controlled release characteristics), it is more preferable case where the composition for a living body according to the embodiment of the present invention contains a water-soluble physiologically active substance as the physiologically active substance, and the composition for a living body according to the embodiment of the present invention undergoes a phase transition from a W/O hexagonal columnar (H2) phase to an O/W hexagonal columnar (H1) phase since the physiologically active substance is accommodated in an interior (W side of W/O) of each column forming the reversed hexagonal columnar (H2) phase, and the physiologically active substance can be released from the interior of the column by the above-described phase transition.

### EXAMPLES

Hereinafter, the present invention will be more specifically described according to Examples, but the present invention is not limited to Examples below.

### Examples 1 to 5 and Comparative Examples 1 to 8

### (Preparation of Substrate)

The substrate was prepared by mixing the components shown in Table 1 in the amounts (parts by mass) shown in the same table.

In Comparative Example 6, Episil® (manufactured by Meiji Seika Pharma Co., Ltd.) was used as the substrate. In the neutral acyl lipid contained in Episil®, the total amount of the neutral acyl lipids is a neutral diacyl lipid (glyceryl dioleate). Since the content of the neutral diacyl lipid in the neutral acyl lipid is more than 50% by mass, Epishell® does not correspond to the composition for a living body according to the embodiment of the present invention.

In Comparative Example 7, Plastibase® (manufactured by Taisho Pharmaceutical Co., Ltd.) was used as the substrate. Plastibase® is a hydrocarbon gel ointment base containing 95% of liquid paraffin and 5% of a polyethylene resin as a gelling agent. Plastibase® does not include the neutral acyl lipid and the phospholipid.

In Comparative Example 8, Macrogol ointment (manufactured by Yoshida Pharmaceutical Co., Ltd.) was used as the substrate. Macrogol ointment is an ointment containing polyethylene glycol 400 and polyethylene glycol 4,000. Macrogol ointment does not contain a neutral acyl lipid and a phospholipid.

### (Preparation of Composition for a Living Body)

Cyanocobalamin (manufactured by FUJIFILM Wako Pure Chemical Corporation) was dispersed in a mortar for each of the above-described substrates to prepare each composition for a living body (sample). The content of the cyanocobalamin was 0.1% by mass with respect to the total amount of each composition for a living body.

### <Evaluation>

The following evaluations were performed using each of the above-described compositions for a living body (sample). The results are shown in Table 1.

### (Controlled Release Characteristics)

### Evaluation Method

A mixture of gelatin (manufactured by FUJIFILM Wako Pure Chemical Corporation) and pure water was put into a container, and mixed and dissolved for 1 hour under conditions of 50° C. using a mix rotor, and then the mixed solution was poured into a mold to produce a gelatin gel (thickness of 7 mm) having a concentration of 23% by mass.

1 g of each of the above-described compositions for a living body prepared as described above was applied onto the above-described gelatin gel in a quadrangular shape of 4.5 cm in length and 1 cm in width, and the mixture was allowed to stand at room temperature (25° C.) and a relative humidity of 50% for 24 hours.

After 24 hours, the composition for a living body was removed from the top of the gelatin gel, and a portion of the gelatin gel (size: 4.5 cm in length, 1 cm in width, and 7 mm in thickness), in which the composition for a living body was removed was put into a measurement cell.

A measurement cell was set in a UV-VIS absorption measuring device (spectrophotometer “V-670” manufactured by JASCO Corporation) such that light was incident on a side surface (surface having a length of 4.5 cm and a thickness of 7 mm) of the gelatin gel.

The film was evaluated for the controlled release characteristics by the following standard using the measured absorbance (abs) at 550 nm obtained by performing the spectrometry.

### Evaluation Criteria

In a case where the absorbance (abs) at 550 nm measured as described above was 0.80 or more, it was evaluated that the obtained film had particularly excellent controlled release characteristics, and this was indicated as “S”.

In a case where the absorbance was 0.10 or more and less than 0.80, it was evaluated that the obtained film had extremely excellent controlled release characteristics, and this was indicated as “A”.

In a case where the absorbance was 0.02 or more and less than 0.10, it was evaluated that the obtained film had slightly excellent controlled release characteristics, and this was indicated as “B”.

On the other hand, in a case where the absorbance was less than 0.02, it was evaluated that the obtained film or the composition for a living body had deteriorated controlled release characteristics, and this was indicated as “C”.

The absorbance at 550 nm measured in the same manner as described above using only the substrates of Examples and Comparative Examples was less than 0.02.

### (Spreadability)

### Evaluation Method

A rheometer device (MCR302) and a measurement jig CP25 were used. Each of the above-described compositions for a living body was set on a base of a rheometer device, and the viscosity of each composition for a living body was measured under the conditions of a measurement temperature of 25° C., 50% rh (relative humidity), and a shear rate (1/s) of 0.1 to 1,000.

### Evaluation Criteria

In a case where the viscosity at shear rate of 0.1 (1/s) was less than 5,000 cPs, it was evaluated that the spreadability (ease of spreading) on the object was extremely excellent, and this was indicated as “A”.

In a case where the above-described viscosity was 5,000 cPs or more and less than 1,000,000 cPs, it was evaluated that the spreadability were slightly excellent, and this was indicated as “B”.

On the other hand, in a case where the viscosity was 1,000,000 cPs or more, it was evaluated that the spreadability was low, and this was indicated as “C”.

### (Film Formation Rate)

### Evaluation Method

Each composition for a living body was applied onto a slide glass to have a thickness of 200 m under conditions of 25° C. and 50% rh.

After the application, the composition for a living body on the slide glass was observed with a polarizing microscope after 1 minute, 30 minutes, 1 hour, and 24 hours.

In the above observation, a time required for the composition for a living body on the slide glass to form a liquid crystal phase (time required for the field of view of the polarizing microscope to change from a dark field to a bright field) was recorded. In a case where the composition for a living body forms a liquid crystal phase, a field of view of a polarizing microscope is a bright field.

### Evaluation Criteria

In a case where the time until the composition for a living body formed a liquid crystal phase was within 1 minute from the application, it was evaluated that the speed of the film formation was very high, and this was indicated as “A”.

Although the above-described A did not apply, in a case where the liquid crystal phase was formed within 30 minutes from the application, it was evaluated that the speed of the film formation was slightly fast, and this was indicated as “B”.

On the other hand, in a case where the liquid crystal phase was formed within 1 hour from the application, which did not correspond to A and B described above, it was evaluated that the rate of the film formation was slightly slow, and this was indicated as “C”.

In a case where the liquid crystal phase was not formed at a point of time 1 hour after the application, which did not correspond to any of A to C described above, it was evaluated that the rate of the film formation was extremely slow, and this was indicated as “D”.

In the evaluation of the present example, in all Examples and Comparative Examples 1 to 6, the liquid crystal phase was formed 24 hours after the application.

In Comparative Examples 7 and 8, the liquid crystal phase was not formed 24 hours after the application. The above results were indicated as “N. D. (non-liquid crystal)”.

### (Strength of Film)

### Evaluation Method

A rheometer device (MCR302) and a measurement jig PP25 were used. Each composition for a living body was applied to the base in advance in a size of a diameter of 25 mm and a thickness of 200 m, and the base was set in a rheometer device. Water was sprayed 3 times (the amount of water sprayed 3 times was 0.3 mL) onto the composition for a living body, which was applied as described above, using a trusco finger spray (TFSB-20), and the composition was allowed to stand for 1 minute.

After 1 minute, the strain dispersion was measured from 0.001% to 1,000% at a measurement temperature of 25° C., a measurement GAP of 200 m, a frequency of 1 Hz, and N_{f}=1 N, and a value of the storage elastic modulus G′ at a shear strain (0.1%) was obtained.

### Evaluation Criteria

In a case where the storage elastic modulus G′ was 15,000 Pa or more, it was evaluated that the strength of the film formed from the composition for a living body was extremely excellent, and this was indicated as “A”.

In a case where the storage elastic modulus G′ was 5,000 Pa or more and less than 15,000 Pa, it was evaluated that the strength of the film was slightly excellent, and this was indicated as “B”.

On the other hand, in a case where the storage elastic modulus G′ was less than 5,000 Pa, it was evaluated that the strength of the film was low, and this was indicated as “C”.

### (Confirmation of Liquid Crystal Phase)

### Evaluation Method

Each of the prepared compositions for a living body was applied onto a slide glass to have a thickness of 200 m, and small-angle X-ray scattering (SAXS) measurement was performed using a sample in which the above-described slide glass had been allowed to stand at 25° C. and 50% rh (relative humidity) for 24 hours or more, and the liquid crystal structure was determined. For the SAXS measurement, a small-angle X-ray scattering measuring device Nanopix (Cu Ka, 40 kV/30 mA) manufactured by Rigaku Corporation was used. The results of the SAXS measurement are shown by a scattering curve using a scattering intensity as the ordinate and a scattering vector length (q/nm⁻¹) as the abscissa. For each peak on the obtained scattering curve, a ratio of the scattering vector length (q/nm⁻¹) was measured to identify the liquid crystal structure.

In a case where at least three peaks were observed on the scattering curve and the ratio of the scattering vector lengths of the peaks was 1:√3:√4, it was determined that the liquid crystal phase was a reversed hexagonal columnar phase. The ratio of the scattering vector lengths of the three peaks of 1:√3:√4 is specific to the reversed hexagonal columnar phase.

### Evaluation Results

In a case where the liquid crystal phase was a reversed hexagonal columnar phase, this was indicated as “H2”.

In a case where the liquid crystal phase was not the reversed hexagonal columnar phase, this was indicated as “not H2”.

As described above, in the evaluation of the present example, in all Examples and Comparative Examples 1 to 6, the liquid crystal phase was formed 24 hours after the application.

In Comparative Examples 7 and 8, since the liquid crystal phase was not formed 24 hours after the application, the above results were indicated as “non-liquid crystal”.

TABLE 1

| | Example 1 | | Example 2 | | Example 3 | | Example 4 | | Example 5 | |
|---|---|---|---|---|---|---|---|---|---|---|
| Neutral acyl lipid 1 | 35 | | 40 | | 45 | | 50 | | | |
| (content of neutral | | | | | | | | | | |
| diacyl lipid in neutral | | | | | | | | | | |
| acyl lipid 1 is 50% by | | | | | | | | | | |
| mass or less, | | | | | | | | | | |
| mono:di:tri = | | | | | | | | | | |
| 49:45:6) | | | | | | | | | | |
| Neutral acyl lipid 2 | | | | | | | | | 45 | |
| (content of neutral | | | | | | | | | | |
| diacyl lipid in neutral | | | | | | | | | | |
| acyl lipid 2 is 50% by | | | | | | | | | | |
| mass or less, | | | | | | | | | | |
| mono:di:tri = | | | | | | | | | | |
| 95:5:0) | | | | | | | | | | |
| (Comparative) Neutral | | | | | | | | | | |
| acyl lipid 3 | | | | | | | | | | |
| (content of neutral | | | | | | | | | | |
| diacyl lipid in neutral | | | | | | | | | | |
| acyl lipid 3 is more | | | | | | | | | | |
| than 50% by mass) | | | | | | | | | | |
| Phospholipid | 65 | | 60 | | 55 | | 50 | | 55 | |
| Propylene glycol | 5 | | 5 | | 5 | | 5 | | 5 | |
| (Comparative) | | | | | | | | | | |
| Oleyl alcohol | | | | | | | | | | |
| Controlled release | A | (0.378) | A | (0.281) | S | (0.850) | B | (0.056) | B | (0.027) |
| characteristics | | | | | | | | | | |
| (absorbance) | | | | | | | | | | |
| Spreadability | B | (20625) | B | (10509) | A | (3172) | A | (1869) | B | (8.67 × 10⁵) |
| (unit: cPs) | | | | | | | | | | |
| Film formation rate | A | (≤1 min) | A | (≤1 min) | A | (≤1 min) | B | (≤30 min) | A | (≤1 min) |
| Strength of film | B | (13362) | A | (18227) | A | (17369) | B | (6197) | B | (8004) |
| (unit: Pa) | | | | | | | | | | |

| | | | | | |
|---|---|---|---|---|---|
| Confirmation of liquid | H2 | H2 | H2 | H2 | not H2 |
| crystal phase | | | | | |

TABLE 2

| | Comparative | Comparative | Comparative | Comparative | Comparative | Comparative | Comparative | Comparative |
|---|---|---|---|---|---|---|---|---|
| Subsequent to Table 1 | Example 1 | Example 2 | Example 3 | Example 4 | Example 5 | Example 6 | Example 7 | Example 8 |
| Neutral acyl lipid 1 | 55 | 60 | 65 | | 45 | Episil^{(R)} | Plastibase^{(R)} | Macrogol |
| (content of neutral diacyl | | | | | | (content of | (containing | ointment^{(R)} |
| lipid in neutral acyl lipid | | | | | | neutral | 95% of liquid | (ointment |
| 1 is 50% by mass or less, | | | | | | diacyl lipid | paraffin and | containing |
| mono:di:tri = 49:45:6) | | | | | | in neutral | 5% of | polyethylene |
| Neutral acyl lipid 2 | | | | | | acyl lipid is | polyethylene | glycol 400 and |
| (content of neutral diacyl | | | | | | more than | resin as | polyethylene |
| lipid in neutral acyl lipid | | | | | | 50% by | gelling | glycol 4,000) |
| 2 is 50% by mass or less, | | | | | | mass) | agent) | |
| mono:di:tri = 95:5:0) | | | | | | | | |
| (Comparative) Neutral | | | | 45 | | | | |
| acyl lipid 3 | | | | | | | | |
| (content of neutral diacyl | | | | | | | | |
| lipid in neutral acyl lipid | | | | | | | | |
| 3 is more than 50% by | | | | | | | | |
| mass) | | | | | | | | |
| Phospholipid | 45 | 40 | 35 | 55 | 55 | | | |
| Propylene glycol | 5 | 5 | 5 | 5 | | | | |
| (Comparative) Oley1 | | | | | 5 | | | |
| alcohol | | | | | | | | |
| Controlled release | C (0.001) | C (0.019) | C (0.012) | C (0) | B (0.097) | B (0.057) | C (0.005) | A (0.12) |
| characteristics | | | | | | | | |
| (absorbance) | | | | | | | | |
| Spreadability | A(1212) | A (711) | A (425) | B (9211) | C | A (94) | B | C |
| (unit: cPs) | | | | | (1.04 × 10⁷) | | (5.70 × 10⁵) | (1.26 × 10⁷) |
| Film formation rate | C (≤1 hour) | D (>1 hour) | D (>1 hour) | A (≤1 min) | A (≤1 min) | C | N.D. | N.D. |
| | | | | | | | (non-liquid | (non-liquid |
| | | | | | | | crystal) | crystal) |
| Strength of film | C (59) | C (0) | C (0) | C (1382) | A (30097) | C (17) | C (3877) | A (47386) |
| (unit: Pa) | | | | | | | | |
| Confirmation of liquid | H2 | H2 | H2 | H2 | H2 | not H2 | Non-liquid | Non-liquid |
| crystal phase | | | | | | | crystal | crystal |

Details of each component in Table 1 are as follows. Details of the substrates used in Comparative Examples 6 to 8 are as described above.

### (Neutral Acyl Lipid)

Neutral acyl lipid 1: a neutral acyl lipid containing a neutral monoacyl lipid, a neutral diacyl lipid, and a neutral triacyl lipid in a mass ratio of 49:45:6, in which the neutral monoacyl lipid contains glyceryl monooleate. Manufactured by Tokyo Chemical Industry Co., Ltd.
Neutral acyl lipid 2: a neutral acyl lipid having a mass ratio between a neutral monoacyl lipid, a neutral diacyl lipid, and a neutral triacyl lipid of 95:5:0, in which the neutral monoacyl lipid contains glyceryl monooleate. GLYMOIST-MO (manufactured by NOF CORPORATION)
(Comparative) Neutral acyl lipid 3: neutral acyl lipid containing neutral monoacyl lipid, neutral diacyl lipid, and neutral triacyl lipid in a mass ratio of 3:78:19. Manufactured by FUJIFILM Wako Pure Chemical Corporation

### (Phospholipid)

Phospholipid: phosphatidylcholine. Lipoid P100 (manufactured by Lipoid GmbH). The content of the phosphatidylcholine was 97.3% by mass in the above product.

### (Solvent)

Propylene glycol (manufactured by Tokyo Chemical Industry Co., Ltd.)
(Comparative) Oleyl alcohol (manufactured by Tokyo Chemical Industry Co., Ltd.)

From the results shown in Table 1, in Comparative Examples 1 to 3 in which the mass ratio (neutral acyl lipid/phospholipid) of the content of the neutral acyl lipid to the content of the phospholipid was out of 54/46 to 1/99, the rate of the film formation was slow, the strength of the obtained film was low, and the controlled release characteristics of the film was deteriorated.

In Comparative Examples 4 and 6 in which the content of the neutral diacyl lipid in the neutral acyl lipid exceeded 50% by mass in the neutral acyl lipid, the strength of the obtained film was low. In Comparative Example 4, furthermore, the controlled release characteristics of the film was deteriorated. In Comparative Example 6, furthermore, the rate of the film formation was slow.

In Comparative Example 5, in which neither the predetermined alcohol nor the polyalkylene oxide was contained and instead oleyl alcohol was contained, the spreadability on the object was low.

In Comparative Examples 7 and 8, in which the neutral acyl lipid and the phospholipid did not contained, a liquid crystal phase (film) was not formed. In Comparative Example 7, furthermore, the controlled release characteristics of the film was deteriorated. In Comparative Example 8, furthermore, the spreadability was low.

On the other hand, the composition for a living body according to the embodiment of the present invention has excellent spreadability on an object, has a high film formation rate in a case of being brought into contact with water to form a film, and has excellent strength and excellent controlled release characteristics of the film.

Among these, from the comparison of Examples 1 to 4, it was confirmed that in a case where the mass ratio of the content of the neutral acyl lipid to the content of the phospholipid was 49/51 to 36/64 (more preferably, 49/51 to 41/59), the effect was more excellent.

In addition, from the comparison between Examples 3 and 5, it was confirmed that, in a case where the content of the neutral monoacyl lipid in the neutral acyl lipid was 40% to 90% by mass, the effect was more excellent.

From the results of the film formation rates of Examples 1 to 4 and Comparative Examples 1 to 3, it was confirmed that the film formation rate tended to be higher as the proportion of the phospholipid in the mass ratio (neutral acyl lipid/phospholipid) was larger.

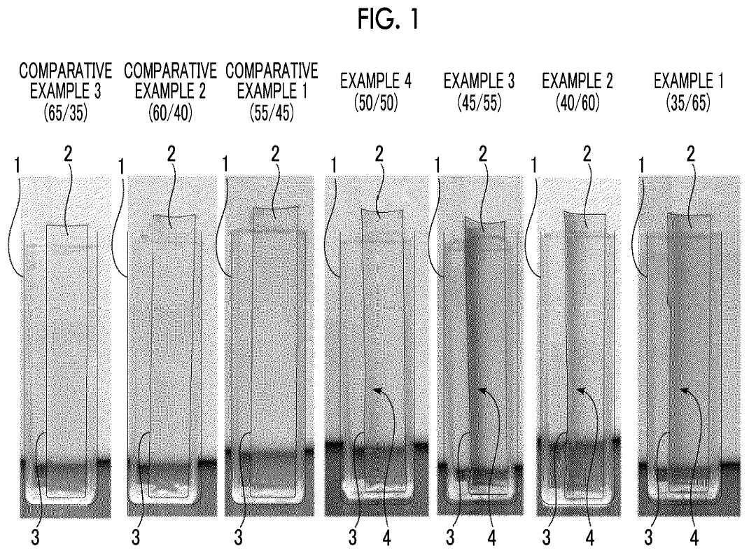

### FIG. 1

FIG. 1 is photographs showing gelatin gels after controlled release of cyanocobalamin from the composition for a living body of Examples 1 to 4 and Comparative Examples 1 to 3 in the evaluation of the controlled release characteristics of the present invention.

In each of Examples and Comparative Examples of FIG. 1, the gelatin gel 2 is accommodated in the measurement cell 1 (the size of the gelatin gel 2 is 4.5 cm in length, 1 cm in width, and 7 mm in thickness). A method of producing the measurement cell 1 accommodating the gelatin gel 2 is as described above in the evaluation method of the controlled release characteristics.

The photograph of FIG. 1 shows a side surface (surface of 4.5 cm in length and 7 mm in thickness) of the gelatin gel 2 of each of Examples and Comparative Examples. Therefore, a surface 3 of the gelatin gel 2 on which the composition for a living body is applied and then removed is shown by a line in FIG. 1.

As shown in FIG. 1, in the gelatin gel 2 of Comparative Example 1, the entire gel was substantially colorless and transparent, and the coloration (red color) due to cyanocobalamin was hardly recognized. The same was applied to Comparative Examples 2 and 3.

On the other hand, in Examples 1 to 4, the colored portion 4 was generated from the surface 3 to the inside of the gelatin gel 2 (the colored portion 4 was colored in red). The colored portion 4 indicates that cyanocobalamin was controlled released from the composition for a living body of Examples 1 to 4, and the sustained-released cyanocobalamin was permeated into the gelatin gel. Among Examples 1 to 4, the color of the colored portion 4 of Example 3 was the darkest. In the gelatin gel 2 of Examples 1 to 4, a portion other than the colored portion 4 was substantially colorless and transparent.

As described above, the film formed of the composition for a living body according to the embodiment of the present invention is excellent in controlled release characteristics.

### EXPLANATION OF REFERENCES

1: measurement cell
2: gelatin gel
3: surface
4: colored portion

What is claimed is:

1. A composition for a living body, comprising:
a neutral acyl lipid containing glyceryl oleate;
a phospholipid;
an alcohol having 4 or less carbon atoms or a polyalkylene oxide; and
a physiologically active substance,
wherein a content of a neutral diacyl lipid in the neutral acyl lipid is more than 0% by mass and 50% by mass or less,
a mass ratio of a content of the neutral acyl lipid to a content of the phospholipid is 54/46 to 1/99, and
the composition for a living body is for percutaneous absorption or mucous membrane absorption.

2. The composition for a living body according to claim 1,
wherein the mass ratio is 54/46 to 30/70.

3. The composition for a living body according to claim 1,
wherein the mass ratio is 49/51 to 36/64.

4. The composition for a living body according to claim 1,
wherein the mass ratio is 49/51 to 41/59.

5. The composition for a living body according to claim 1,
wherein the neutral acyl lipid further contains a neutral monoacyl lipid, and
a content of the neutral monoacyl lipid in the neutral acyl lipid is 40% to 90% by mass.

6. The composition for a living body according to claim 5,
wherein the neutral monoacyl lipid contains glyceryl monooleate.

7. The composition for a living body according to claim 1,
wherein the phospholipid contains an ionic phospholipid.

8. The composition for a living body according to claim 1,
wherein the phospholipid contains phosphatidylcholine, and
a content of the phosphatidylcholine in the phospholipid is at least 50% by mass.

9. The composition for a living body according to claim 1,
wherein a content of the alcohol having 4 or less carbon atoms or the polyalkylene oxide is 0.5 to 10 parts by mass with respect to 100 parts by mass of a total content of the neutral acyl lipid and the phospholipid.

10. The composition for a living body according to claim 1,
wherein a content of water is 0% by mass or more and 10% by mass or less with respect to a total amount of a remainder obtained by removing the physiological active substance from the composition for a living body.

11. The composition for a living body according to claim 1,
wherein a reversed hexagonal columnar phase is formed by water absorption or moisture absorption.

12. The composition for a living body according to claim 2,
wherein the mass ratio is 49/51 to 36/64.

13. The composition for a living body according to claim 2,
wherein the mass ratio is 49/51 to 41/59.

14. The composition for a living body according to claim 2,
wherein the neutral acyl lipid further contains a neutral monoacyl lipid, and
a content of the neutral monoacyl lipid in the neutral acyl lipid is 40% to 90% by mass.

15. The composition for a living body according to claim 14,
wherein the neutral monoacyl lipid contains glyceryl monooleate.

16. The composition for a living body according to claim 2,
wherein the phospholipid contains an ionic phospholipid.

17. The composition for a living body according to claim 2,
wherein the phospholipid contains phosphatidylcholine, and
a content of the phosphatidylcholine in the phospholipid is at least 50% by mass.

18. The composition for a living body according to claim 2,
wherein a content of the alcohol having 4 or less carbon atoms or the polyalkylene oxide is 0.5 to 10 parts by mass with respect to 100 parts by mass of a total content of the neutral acyl lipid and the phospholipid.

19. The composition for a living body according to claim 2,
wherein a content of water is 0% by mass or more and 10% by mass or less with respect to a total amount of a remainder obtained by removing the physiological active substance from the composition for a living body.

20. The composition for a living body according to claim 2,
wherein a reversed hexagonal columnar phase is formed by water absorption or moisture absorption.
